(19) United States
(12) Patent Application Publication
(10) Pub. No.: US 2021/0046225 A1
(43) Pub. Date: Feb. 18, 2021

(54) METHOD OF HARVESTING AND PROCESSING SOFT TISSUE AND KIT THEREFOR

(71) Applicants: David Karol Angeloni, JR., Carlsbad, CA (US); Gregory Mark Miles, Carlsbad, CA (US)

(72) Inventors: David Karol Angeloni, JR., Carlsbad, CA (US); Gregory Mark Miles, Carlsbad, CA (US)

(21) Appl. No.: 15/929,424

(22) Filed: May 1, 2020

Related U.S. Application Data
(60) Provisional application No. 62/816,324, filed on Mar. 11, 2019.

(51) Int. Cl. A61M 1/00

(52) U.S. Cl. CPC A61M 1/0094; A61M 1/007; A61M 2205/7545; A61M 2202/08; A61M 1/008

(57) ABSTRACT

A method for autologous soft tissue transfer and a single use surgical kit are provided. The method includes harvesting soft tissue with a tumescent fluid and filtering the harvested soft tissue to obtain a drained aspirate. The single use surgical kit includes at least one syringe, at least one infiltration cannula, at least one aspiration cannula, and at least one tissue filter. The infiltration cannula has a spiral hole array.

### CROSS-REFERENCE TO RELATED APPLICATION

This application claims the benefit of priority of U.S. provisional application No. 62/816,324, filed Mar. 11, 2019, the contents of which are herein incorporated by reference.

### BACKGROUND OF THE INVENTION

The present invention relates to autologous soft tissue transfer and, more particularly, to a method and kit for improved tissue harvesting and processing during transfer.

Autologous soft tissue transfer, especially adipose, is currently performed with devices and processes that are time-consuming and burdensome and that damage the tissues being grafted. Many of the tools used to harvest or process soft tissue cause duress to the cells and tissues. Processes to emulsify and/or micronize tissue cause unwanted damage. These disadvantages may lead to undesirable surgical outcomes including infection, micro calcifications, and general graft failure. Many current tissue transfer methods require post processing sets that can be time-consuming and cost prohibitive, and cause damage to the graft, leading to diminished outcomes.

As can be seen, there is a need for a quick, easy process for autologous soft tissue transfer that does not damage graft tissue. There is also a need for devices to achieve a quick, easy, nondamaging soft tissue transfer process.

The present invention provides a shortened method of harvesting, resizing, and transferring soft tissue while maintaining cell and tissue viability and integrity by reducing damage to the tissue and cells. The method may further avoid transferring unwanted cells and byproducts of the procedure to the target site. Another aspect of the invention provides a single use surgical kit for autologous soft tissue transfer.

The inventive kit and the inventive method improve upon three main characteristics of soft tissue harvest and transfer. First, the kit may comprise preselected cannulas and filters that limit both the positive and negative pressure to which the tissue and cells are exposed during the procedure. The cannulas and filters thereby improve viability and integrity of the surgical graft, potentially improving surgical outcomes. Second, washing, centrifuging, emulsifying, and resizing are generally unnecessary when harvesting soft tissue with the inventive kit and may be avoided, saving time. Third, removing these unnecessary steps may improve outcomes and save both overhead costs and costs associated with possible surgical revisions.

### SUMMARY OF THE INVENTION

In one aspect of the present invention, a method for autologous soft tissue transfer is provided, comprising infiltrating a tumescent fluid into the soft tissue; harvesting soft tissue with the tumescent fluid; filtering harvested soft tissue to obtain a drained aspirate; and grafting the drained aspirate.

In another aspect of the present invention, a single use surgical kit is provided, comprising at least one syringe, at least one infiltration cannula, at least one aspiration cannula, and at least one tissue filter, wherein the at least one infiltration cannula has a spiral hole array.

These and other features, aspects and advantages of the present invention will become better understood with reference to the following drawings, description and claims.

### BRIEF DESCRIPTION OF THE DRAWINGS

FIG. 1 is an exploded view of a kit according to an embodiment of the invention:

FIGS. 2A, 2B, 3, 4, 5A, 5B, 6, 7, 8A, 8B, 9, and 10 are schematic views illustrating components of the kit of FIG. 1 arranged for use in a series of steps according to an embodiment of the invention;

FIG. 11 is a side perspective view of a cannula used with a power-assisted liposuction instrument in the method thereof;

FIG. 12 is a right-side elevation view thereof, the left-side elevation being the same view;

FIG. 13 is a top plan view thereof, the bottom view being the same view;

FIG. 14 is a front elevation view thereof; and

FIG. 15 is a rear elevation view thereof.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

As used herein, the term “soft tissue” refers to tissues that connect, support, or surround other structures and organs of the body, including muscles, tendons, ligaments, fascia, fibrous tissues, fat, blood vessels, and synovial membranes.

Broadly, one embodiment of the present invention is a method for harvesting and processing soft tissue. Another embodiment of the invention is a single use surgical kit therefor.

The present invention includes a method, using liposuction harvesting cannula to harvest and prepare soft tissue, like adipose, for collection and grafting purposes. The harvesting cannulas and the resizer used are dependent upon the procedure or the desired product. One may achieve the desired adipose size clusters via the cannulas but if these cannulas struggle to harvest the smaller particle size (e.g., 1.0 mm or less), resizers may achieve these smaller sizes without damaging the soft tissue and cells within. Filters may be used to clean out excess fluid which contains unwanted blood, lipids, and/or drugs so that the small tissue clusters and desired cells are not lost, and the tissue may be maintained within a closed system to maintain sterility.

In some aspects of the invention, a support apparatus for storing syringes used for transfer in an upright arrangement may be provided. A method of placing the syringes in an upright arrangement may also be provided.

The liposuction cannula may harvest viable soft tissue clusters of various sizes in a particular array in-vivo. As such, steps meant to emulsify and/or micronize such tissue in order to inject the tissue via small transfer cannulas or needles are generally unnecessary.

The inventive method comprises a tumescent harvesting technique whereby the cannulas may be used to achieve a particular size tissue cluster. The cannula sizes may include but are not limited to a 2.0 mm aperture and a 1.0 mm aperture, both of which may contain a 3 to 24+ hole configuration/array, and a 500 μm aperture with a hole array surrounding the cannula ranging from 50-1000+ hole array, such as about a 300-hole array.

In some embodiments, the method also includes a filtration step to remove unwanted debris and byproducts that allows the capture of viable cells typically lost via other tissue washing and processing methods. These lost cells may then be added back to the harvested tissue in order to maintain a more comprehensive graft.

Since every patient is different from an anatomic standpoint and because each procedure has unique requirements, each system contains redundancies to assure physician and patient satisfaction. One of these redundancies may be the ability to break down tissue in the event the practitioner has difficulty harvesting the desired particle size. It is typically easier to harvest larger size tissue particles with larger size cannulas and apertures.

The size of the tissue particles created is important for efficacy of the graft. Without the appropriate size particle, the surgery may not get the intended result. The method and device improve upon both as the cannulas maximize graft survival during the harvest via the hole and aperture configuration. The cannulas allow a physician to harvest the graft size required in a time-efficient and viable manner.

If the physician wants a micronized soft tissue graft for specific applications, the graft tissue may be resized. This micronized tissue may be referred to as tissue stromal vascular fraction (tSVF), nanofat, or “stromal vascular tissue” (SVT). Harvested tissue may be further downsized during a medical procedure. For example, the physician may harvest 1.0 mm size tissue clusters (raw product) from the patient for facial volumization but requires stromal vascular tissue to remodel some areas of the skin where volume isn't required, only repair for skin related issues such as sun damage, rhytids, hyperpigmentation, etc. The physician may then pass about 5 ml-about 60 ml of raw product through a micronizer, such as the 500 μm Diamond Cut™ resizer, between two syringes until the product is homogenized and uniform in consistency and color. This raw micronized tissue product may then be washed and filtered.

In some cases, harvested graft or processed product may be filtered due to potential contaminants within the graft (lipoaspirate) which may include one or more of blood, lipids, and drugs like lidocaine and/or epinephrine. The processed product may be passed through a mesh filter having a mesh size that may range from about 50 μm to about 1000 μm to remove unwanted debris, blood cells, lipids, and drugs within the tumescent. Then with a female luer to luer syringe connector, an equal amount of the tissue product (SVT) may be added to an equal amount of sterile saline and then filtered with the same filter as in the previous step or with a new filter. The now-clean tissue may be parceled into desired transfer syringes, several of which may be contained within the kit for tissue grafting.

The inventive method may use specialized single use kits packaged and sterilized together. These inventive kits may be structured by individual components based on the desired procedure. The components may be any component selected from the group consisting of: a disposable infiltration cannula with spiral hole array, one or more disposable harvesting cannulas, a syringe adapter, one or more female luer connectors, one or more tissue filters, and combinations thereof.

The kit comprises at least three elements: infiltration and aspiration cannulas; a means of further micronization (sizing) of the tissue; and at least one filter to remove unwanted fluid or waste from the harvested graft. The filters may vary depending upon the other kit components present and the desired graft size.

In some embodiments, depending upon the surgical application, the inventive kit may be configured for harvesting, processing and transferring tissue (adipose) and may include components selected from the group consisting of a 0.5 mm-5 mm aperture harvesting cannula, a 200-1000 μm micronizer, a mesh filter sized for filtering 100-900 μm or smaller particles, and a combination thereof.

In some embodiments, a filter housing may be provided to house a filter for washing the adipose or other soft tissue.

In some embodiments, the kit may comprise components selected from the group consisting of one or more transfer cannulas in preselected sizes, one or more syringe clips (e.g., VacLok®), one or more luer caps, and combinations thereof.

One suitable harvesting cannula is taught by U.S. Design Pat. No. D864,381 S to Miles, which discloses a liposuction cannula which may harvest viable soft tissue clusters of various sizes in a particular array in-vivo. The disclosure of D864,381 S is incorporated herein by reference. Using the Miles cannula, also referred to herein as the “SuperG® Micro cannula”, it is not necessary to emulsify or micronize the tissue in order to inject the tissue via small transfer cannulas or needles for certain medical procedures. The Miles cannula contains 500 μm apertures in a 300-hole array.

The infiltration cannula may be characterized by a spiral hole array. Sizes may range from about 1.0 mm-about 2.5 mm diameter and about 15 cm-about 30 cm long.

Syringe adapters and luer connectors may have 3-10 mm cannula thread with a cannula support. Syringe adapters may be manufactured from stainless steel material machined on the lathe.

The tissue filters may filter particles from about 5 μm to about 1000 μm. The filter housing diameters may range from about 5 mm to about 100 mm. The filters and housing may be injected molded.

A disposable harvesting cannula may be manufactured of stainless-steel tube cut to a predetermined length, swaged, and machined with a preselected hole configuration. The holes may be polished to minimize sharp edges. Passivation may provide the surfaces with reduced reactivity. The cannula hub may be made of a disposable plastic on a lath or in a mold and may be provided with a hole associated with the cannula size. The cannula inserted into the hub may be held in place with adhesive. Sizes may range from about 1.0 m. diameter to about 5.0 mm diameter, in lengths from about 3 cm long to about 35 cm long with hole array ranging from about 100 μm to about 5 mm diameter aperture. Disposable microfacial transfer cannulas may be provided ranging from about 28 g to about 12 g with lengths of about 3 cm to about 15 cm long. The tip of the cannula may be swaged to have a bullet nose shape, then machined to form a cup configuration. The cannula may be marked with depth measurements.

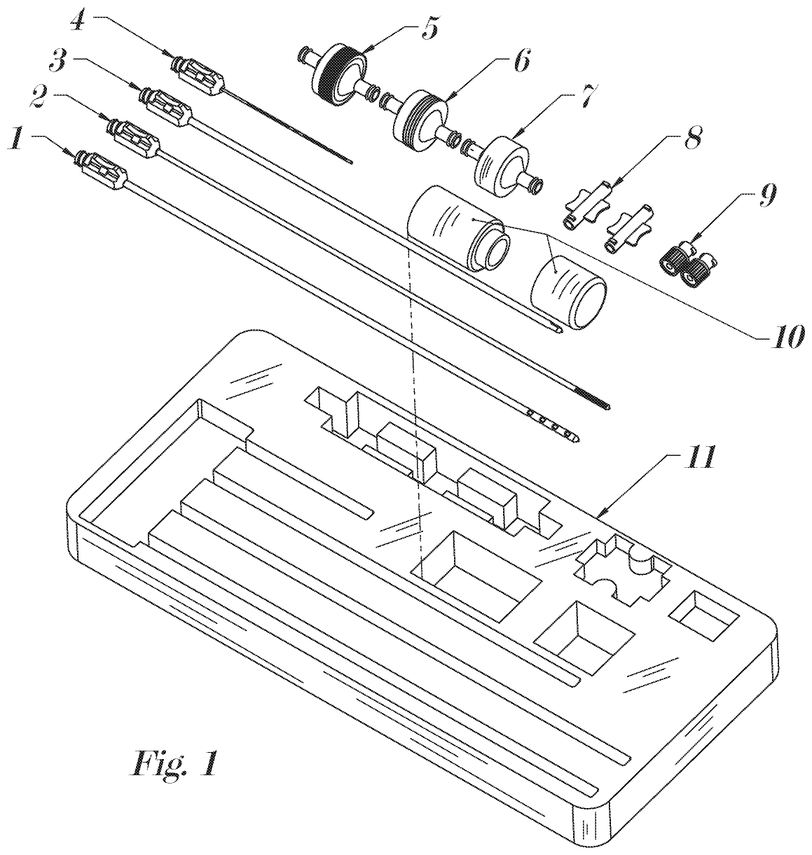

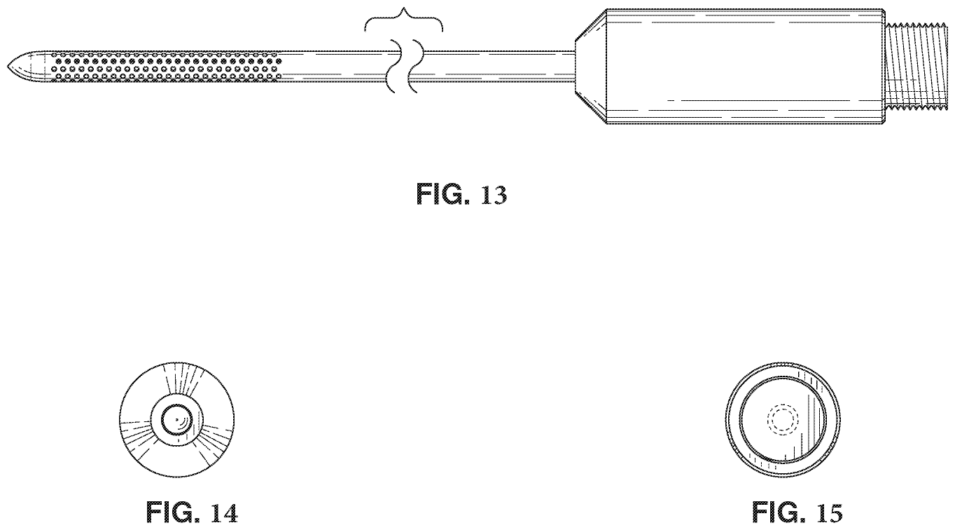

Referring to FIGS. 1-15, FIG. 1 illustrates a kit according to an embodiment of the present invention. The kit may include components selected from the group consisting of: a Milli Cannula 1, a Micro Cannula or Infiltration Cannula 2, one or more Transfer Cannulas 3 which may be provided in various sizes, one or more Transfer Cannulas 4 which may be provided in various sizes, a Millifat Filter 5, a Microfat Filter or Cell Capture Filter 6, a Diamond Cut™ Resizer 7, a Female to Female Luer connector 8, one or more Syringe Caps 9, a Stroma Stamp 10, and combinations thereof.

FIGS. 2A through 10 illustrate a process according to an embodiment of the invention. FIG. 2A illustrates an infiltration cannula 1 and a syringe 12. A provider may fill the syringe 12 with tumescent fluid, as shown in FIG. 2B, and assemble the syringe 12 with the appropriately sized cannula 1. The provider may the infiltrate tumescent fluid into soft tissue, like adipose, with the cannula 1 and, if necessary, an adapter (not shown) which may be provided in the kit.

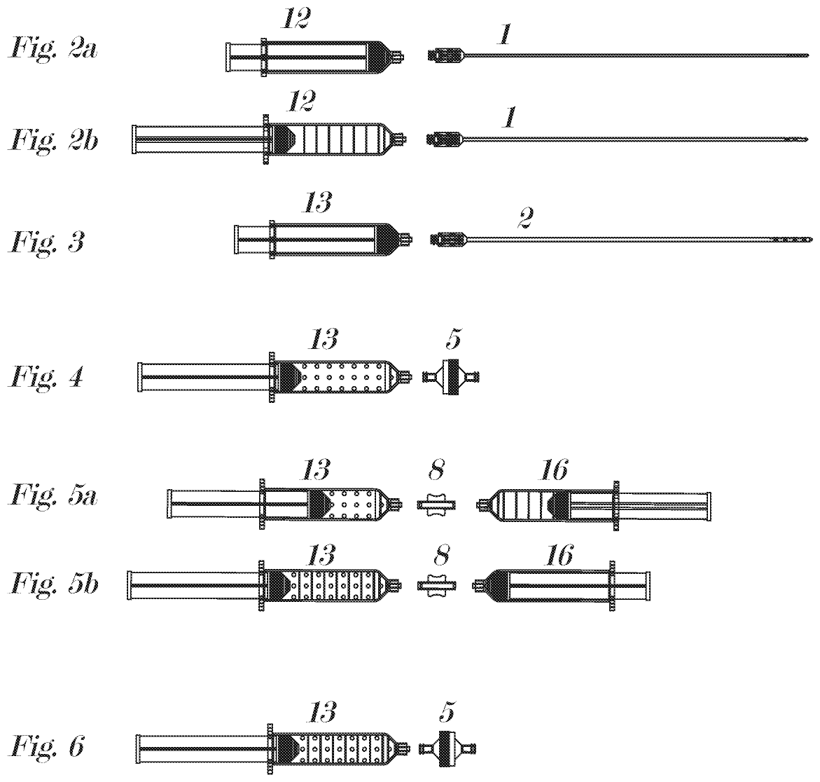

The provider may use a syringe clip (not shown) to create negative pressure within a sterile syringe 13. The provider may attach a desired aspiration (i.e., harvest) cannula 2 to the syringe 13, as shown in FIG. 3, and may harvest a desired volume of tissue. These steps may be repeated until the desired volume and soft tissue size particle has been achieved. The harvest cannula 2 may then be removed, and the tissue-containing syringe 13 may be placed onto a vertical syringe stand for gravity separation (not shown). When the provider is ready, the syringe may be removed from the vertical stand.

A filter 5 may be selected from the kit based upon the size of tissue harvested and attached to the tissue-containing syringe 13, as shown in FIG. 4. Waste material may be ejected from the syringe 13 through the filter 5, leaving drained aspirate in the syringe 13.

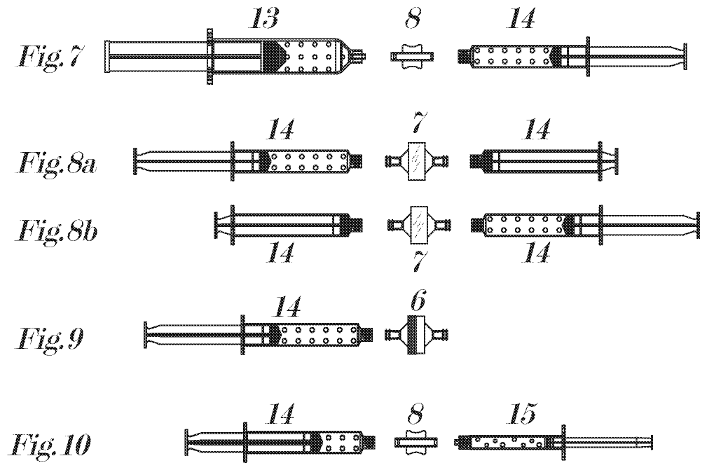

In some cases, the aspirate may be washed. As shown in FIG. 5A, the syringe 13 containing drained aspirate may be attached via a female to female luer connector 8 to a syringe 16 filled with sterile saline. The saline may be ejected into the aspirate-containing syringe 13 and the mixed aspirate-saline suspension may be repeatedly moved between syringes 13, 16 to ensure mixing until the mixed aspirate-saline suspension is held within the syringe 13 as shown in FIG. 5B. FIG. 6 illustrates attaching the other side of the milli filter 5 to syringe 13, allowing the provider to eject waste from the washed aspirate. As shown in FIG. 7, the washed and filtered aspirate may be transferred from the syringe 13 to a smaller syringe 14 via a female to female luer connector 8.

If a predetermined particle size is not achieved during harvest, the filtered tissue may be micronized to a desired size with a supplied sizer 7, as shown in FIG. 8A, with an empty syringe 14 attached to other side of the sizer 7. Tissue may then be passed back and forth between the syringes 14 until a desired size is achieved, as shown in FIG. 8B. The syringe 14 with tissue may be placed on the vertical stand (not shown) again and removed when the provider is ready to attach an appropriate supplied filter 6, as shown in FIG. 9.

As shown in FIG. 10, the aspirate, which may have been filtered, drained, and sized, may be transferred via a luer connector 8 to a desired syringe 15 for transfer to the patient by way of a desired transfer/grafting method.

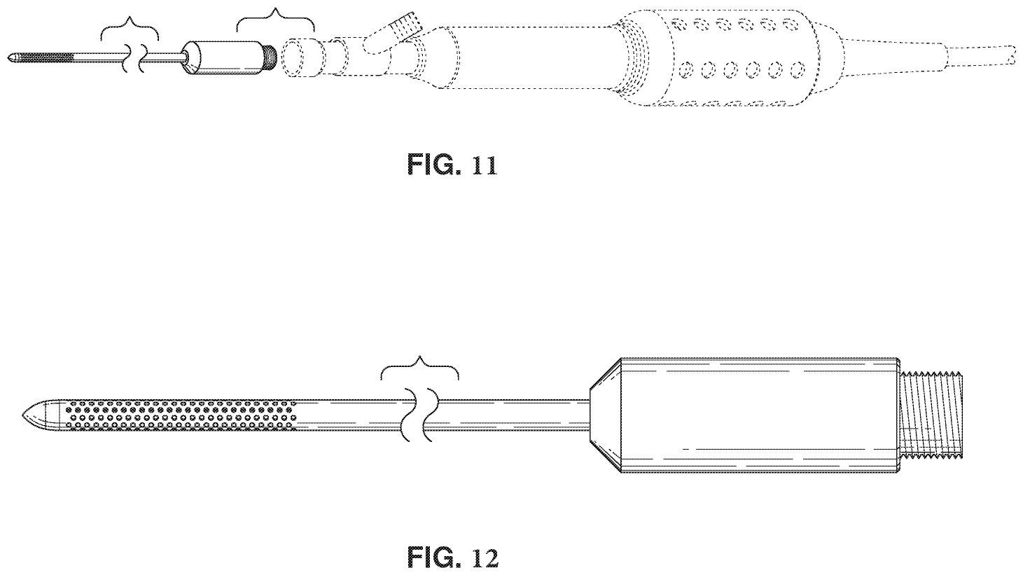

FIGS. 11-15 illustrate one type of cannula appropriate for use with the inventive method and kit. In some embodiments, the cannula of FIG. 11 may be used with a power assist device (not shown), such as an AcquiCell™ system or a Euromi™ Lipomatic system, allowing the user to harvest soft tissue more quickly. In these cases, the power assist device may replace the sterile syringe 13 shown in

FIG. 3.

It should be understood, of course, that the foregoing relates to exemplary embodiments of the invention and that modifications may be made without departing from the spirit and scope of the invention as set forth in the following claims.

What is claimed is:

1. A method for autologous soft tissue transfer, comprising:
a. harvesting soft tissue with a tumescent fluid; and
b. filtering harvested soft tissue to obtain a drained aspirate.

2. The method of claim 1, further comprising a step selected from the group consisting of: infiltrating the tumescent fluid into the soft tissue; washing the drained aspirate with an equal amount of sterile saline; filtering the washed drained aspirate to obtain a washed aspirate; resizing the drained aspirate to produce stromal vascular tissue; grafting the drained aspirate; and combinations thereof.

3. A single use surgical kit, comprising at least one syringe, at least one infiltration cannula, at least one aspiration cannula, and at least one tissue filter, wherein the at least one infiltration cannula has a spiral hole array.

4. The single use surgical kit of claim 3, further comprising a component selected from the group consisting of: a micronizer; a female luer connector; at least one syringe adapter; a filter housing; at least one transfer cannula; at least one syringe clip; at least one luer cap; a stroma stamp; and combinations thereof.

5. A method for autologous soft tissue transfer, comprising utilizing the single use surgical kit of claim 3 to harvest and filter soft tissue.
